(19) United States
(12) Patent Application Publication
(10) Pub. No.: US 2026/0215847 A1
(43) Pub. Date: Jul. 30, 2026

(54) SYSTEM FOR OPTIC-BASED FILTERED TELEMETRY MEASUREMENTS

(71) Applicant: Covidien LP, Mansfield, MA (US)

(72) Inventors: John W. KOMP, Dillon, CO (US); Scott E.M. FRUSHOUR, Boulder, CO (US); William J. DICKHANS, Longmont, CO (US)

(21) Appl. No.: 19/145,543

(22) PCT Filed: Jan. 5, 2024
(86) PCT No.: PCT/IB2024/050114

§ 371 Date: Jul. 3, 2025

Related U.S. Application Data
(60) Provisional application No. 63/438,591, filed on Jan. 12, 2023.

(51) Int. Cl. A61B 18/18; A61B 18/00

(52) U.S. Cl. CPC A61B 18/1815; A61B 2018/00023; A61B 2018/00577; A61B 2018/00791; A61B 2018/1823; A61B 2018/1861

(57) ABSTRACT

An ablation system includes an ablation device configured to ablate a target, and a computing device. The ablation device includes fiber Bragg gratings for monitoring temperature. The computing device is configured to calculate temperature measurements based on light reflected from the fiber Bragg gratings during application of microwave ablation energy to the target and filter the temperature measurements to remove noise.

### CROSS-REFERENCE TO RELATED APPLICATIONS

This application claims the benefit of U.S. Provisional Patent Application Ser. No. 63/438,591, filed Jan. 12, 2023, the entire content of which is incorporated herein by reference.

### FIELD

The present disclosure relates to systems, methods, and devices for generating optic-based noise filtered telemetry measurements.

### BACKGROUND

When planning a treatment procedure, clinicians often rely on patient data including X-ray data, computed tomography (CT) scan data, magnetic resonance imaging (MRI) data, or other imaging data that allows the clinician to view the internal anatomy of a patient. The clinician utilizes the patient data to identify targets of interest and to develop strategies for accessing the targets of interest for a treatment procedure such as a microwave ablation treatment procedure.

Microwave antennas generate heat in the body to provide hyperthermic temperatures to destroy tissues of interest. A challenge with radiated fields that are emitted from a high-frequency antenna is that it is difficult to measure the temperature profile induced in the tissue resulting from exposure to the radiated field, thus complicating the ability to determine the zone of ablation during the application of energy to the tissue.

Existing optical temperature measuring systems generate noisy signals that produce inaccurate temperature measurements.

### SUMMARY

Systems and methods for generating optic-based noise filtered telemetry measurements are provided.

According to an aspect of the present disclosure, an ablation system includes an ablation device, a cooling system, and a computing device. The ablation device includes a radiating antenna configured to apply microwave ablation energy to a target within a patient to ablate the target, a cable extending from the radiating antenna and configured to connect the radiating antenna to a microwave generator, a first fiber including first Bragg gratings formed along a length of the first fiber proximate to the radiating antenna and configured to reflect light therefrom, a second fiber including second Bragg gratings formed along a length of the second fiber and longitudinally spaced apart from the first Bragg gratings along a longitudinal axis of the ablation device and configured to reflect light therefrom. The cooling system includes a pump configured to pump a cooling fluid to the ablation device to cool the ablation device. The computing device includes a processor and memory storing instructions which, when executed by the processor, cause the computing device to calculate first telemetry measurements associated with the radiating antenna based on the reflected light from the first Bragg gratings, calculate second telemetry measurements associated with the cooling system based on the reflected light from the second Bragg gratings, and filter the first telemetry measurements based on the second telemetry measurements.

In an aspect, the computing device may be configured to filter the first telemetry measurements by subtracting the second telemetry measurements from the first telemetry measurements.

In an aspect, the second telemetry measurements may correspond to noise generated by the pump of the cooling system.

In an aspect, the first Bragg gratings may be etched into the first fiber and the second Bragg gratings may be etched into the second fiber.

In an aspect, the ablation device may be a flexible microwave ablation catheter configured to be navigated through a patient's luminal network.

In an aspect, the ablation device may be a rigid microwave ablation device configured to be percutaneously inserted through tissue.

In an aspect, the first telemetry measurements may correspond to a temperature of the radiating antenna and the second telemetry measurements may correspond to motion of the cable.

In accordance with another aspect of the disclosure, an ablation system includes an ablation device, a cooling system, and a computing device. The ablation device includes a radiating antenna configured to apply microwave ablation energy to a target within a patient to ablate the target, a cable extending from the radiating antenna and configured to connect the radiating antenna to a microwave generator, and a fiber. The fiber includes first Bragg gratings formed along a length of the fiber proximate the radiating antenna and configured to reflect light therefrom, and second Bragg gratings formed along a length of the fiber and spaced apart from the first Bragg gratings and configured to reflect light therefrom. The cooling system includes a pump configured to pump a cooling fluid to the ablation device to cool the ablation device. The computing device includes a processor and memory storing instructions which, when executed by the processor, cause the computing device to calculate first telemetry measurements associated with the radiating antenna based on the reflected light from the first Bragg gratings, calculate second telemetry measurements associated with the cooling system based on the reflected light from the second Bragg gratings, and filter the first telemetry measurements based on the second telemetry measurements.

In an aspect, the computing device may be configured to filter the first telemetry measurements by subtracting the second telemetry measurements from the first telemetry measurements.

In an aspect, the second telemetry measurements may correspond to noise generated by the pump of the cooling system.

In an aspect, the first Bragg gratings and the second Bragg gratings may be etched into the fiber.

In an aspect, the ablation device may be a flexible microwave ablation catheter configured to be navigated through a patient's luminal network.

In an aspect, the ablation device may be a rigid microwave ablation device configured to be percutaneously inserted through tissue.

In an aspect, the first telemetry measurements may correspond to a temperature of the radiating antenna and the second telemetry measurements may correspond to motion of the cable.

In accordance with another aspect of the disclosure, a method for filtering telemetry measurements includes receiving reflected light from first Bragg gratings and reflected light from second Bragg gratings, calculating first telemetry measurements associated with a radiating antenna based on the reflected light from the first Bragg gratings, calculating second telemetry measurements associated with a cooling system based on the reflected light from the second Bragg gratings, and filtering the first telemetry measurements based on the second telemetry measurements.

In an aspect, filtering the first telemetry measurements based on the second telemetry measurements may include subtracting the second telemetry measurements from the first telemetry measurements.

In an aspect, receiving reflected light from first Bragg gratings and reflected light from second Bragg gratings may include receiving reflected light from first Bragg gratings formed in a first fiber and receiving reflected light from second Bragg gratings formed in a second fiber.

In an aspect, calculating first telemetry measurements associated with a radiating antenna based on the reflected light from the first Bragg gratings may include calculating a temperature of the radiating antenna.

In an aspect, calculating second telemetry measurements associated with a cooling system based on the reflected light from the second Bragg gratings may include calculating motion induced by a pump of the cooling system.

In an aspect, the method may further include generating an ablation zone volume based on the filtered measurements and displaying the ablation zone volume on a display.

Any of the above aspects and embodiments of the present disclosure may be combined without departing from the scope of the present disclosure.

### BRIEF DESCRIPTION OF THE DRAWINGS

Objects and features of the presently disclosed system and method will become apparent to those of ordinary skill in the art when descriptions of various embodiments thereof are read with reference to the accompanying drawings, of which:

FIG. 1 is a schematic diagram of a microwave ablation system in accordance with an illustrative aspect of the present disclosure;

FIG. 2 is a schematic diagram of a computing device which forms part of the microwave ablation system of FIG. 1 in accordance with an aspect of the present disclosure;

FIG. 3A is a cross-sectional view of an ablation device having a single fiber in accordance with an aspect of the present disclosure;

FIG. 3B is a cross-sectional view of an ablation device having two fibers in accordance with an aspect of the present disclosure; and

FIG. 4 is a flowchart illustrating a method for generating optic-based, noise-filtered telemetry measurements in accordance with an aspect of the present disclosure.

### DETAILED DESCRIPTION

The present disclosure provides a system and method for simulating and displaying ablation zones in real time in a microwave ablation treatment procedure using filtered optic-based temperature measurements. In particular, the present disclosure provides an ablation device including fiber Bragg gratings for non-contact inferential sensing of various parameters including temperature, strain, pressure, torque and more and filtering the temperature measurements to remove noise in the signals. The gratings are embedded via laser etching inside a glass fiber with an explicitly known grating profile associated with the Bragg wavelength or frequency. Exposure to temperature and/or strain causes a broad-spectral pulse of light to reflect off the Bragg grating and shift from the center frequency either up or down in frequency at a known coefficient related to the parameter of interest. In some aspects, the methods and system may be utilized to ensure the system is working as desired or intended based on a comparison of an expected or known acceptable noise baseline and a measured noise value.

Incorporating fiber Bragg gratings into a microwave ablation device enables real-time parameter sensing, for example real-time temperature sensing, which can be utilized to inform the treating physician of the dimensions of ablation volume being generated in real time as the application of ablation energy progresses.

Although the present disclosure will be described in terms of specific illustrative embodiments, it will be readily apparent to those skilled in the art that various modifications, rearrangements, and substitutions may be made without departing from the spirit of the present disclosure. The scope of the present disclosure is defined by the claims appended hereto.

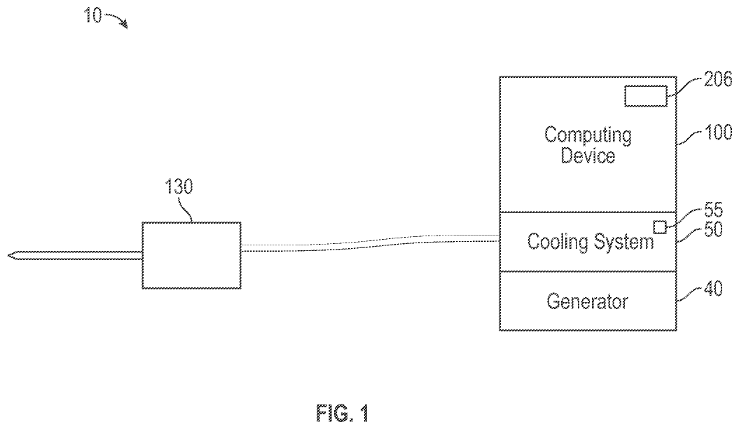

FIG. 1 depicts an ablation system 10 including an ablation device 130, a computing device 100, a cooling system 50, and a microwave generator 40. The ablation device 130 is configured to couple to the microwave generator 40 for transmission of microwave energy generated by the microwave generator 40 to a target within a patient. The computing device 100 may include the cooling system 50 and/or the microwave generator 40 or each may be separate components. The cooling system 50 includes a pump 55 which is configured to pump a cooling fluid to the ablation device 130 to cool the ablation device 130 during operation thereof.

Ablation device 130 is a surgical instrument having a microwave ablation antenna that is used to ablate tissue, such as a lesion or tumor, by using electromagnetic radiation or microwave energy to heat tissue in order to denature or kill cancerous cells. Ablation device 130 may be a flexible microwave ablation catheter configured to be navigated to a target via a patient's luminal network (e.g., through a catheter that has been navigated to the target). Alternatively, ablation device 130 may be a rigid microwave ablation device configured to be percutaneously inserted through tissue to access a target within a patient. Ablation device 130 is configured to connect to microwave generator 40 (FIG. 1) which generates and controls the application of microwave energy through the ablation device 130. Microwave generator 40 may be a component of computing device 100 or may be a separate stand-alone component.

Ablation system 10 may be an Electromagnetic Navigation (EMN) system configured for reviewing CT image data to identify one or more targets, planning a pathway to an identified target (planning phase), navigating a catheter (e.g., an extended working channel) of a catheter guide assembly to a target (navigation phase) via a user interface, and confirming placement of the catheter relative to the target. One such EMN system is the ELECTROMAGNETIC NAVIGATION BRONCHOSCOPY® system currently sold by Medtronic plc. The target may be tissue of interest identified by review of the CT image data during the planning phase. Following navigation, a medical instrument, such as a biopsy tool, ablation tool (e.g., ablation device 130), or other tool, may be inserted into the catheter to obtain a tissue sample (or perform any treatment) from the tissue located at, or proximate to, the target.

Computing device 100 may be any suitable computing device including a processor and storage medium, wherein the processor is capable of executing instructions stored on the storage medium. The computing device 100 may further include a database configured to store patient data, CT data sets including CT images, fluoroscopic data sets including fluoroscopic images and video, navigation plans, and any other such data. Although not explicitly illustrated, the computing device 100 may include inputs, or may otherwise be configured to receive, CT data sets, fluoroscopic images/video and other data described herein. Additionally, computing device 100 includes a display (e.g., display 206) configured to display graphical user interfaces.

With respect to a planning phase, computing device 100 utilizes previously acquired CT image data for generating and viewing a three-dimensional model or rendering of patient “P's” airways, enables the identification of a target on the three-dimensional model (automatically, semi-automatically, or manually), and allows for determining a pathway through patient “P's” airways to tissue located at and around the target. More specifically, CT images acquired from previous CT scans are processed and assembled into a three-dimensional CT volume, which is then utilized to generate a three-dimensional model of patient “P's” airways. The three-dimensional model may be displayed on a display 206 associated with computing device 100, or in any other suitable fashion. Using computing device 100, various views of the three-dimensional model or enhanced two-dimensional images generated from the three-dimensional model are presented. The enhanced two-dimensional images may possess some three-dimensional capabilities because they are generated from three-dimensional data. The three-dimensional model may be manipulated to facilitate identification of target on the three-dimensional model or two-dimensional images, and selection of a suitable pathway through patient “P's” airways to access tissue located at the target can be made. Once selected, the pathway plan, three-dimensional model, and images derived therefrom, can be saved and exported to a navigation system for use during the navigation phase(s). One such planning software is the ILLUMISITE® planning suite currently sold by Medtronic plc.

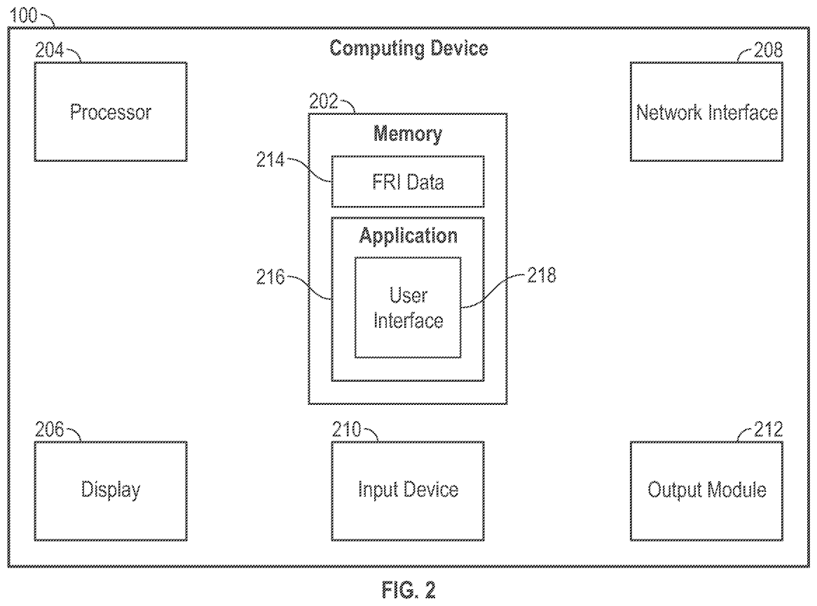

FIG. 2 illustrates a system diagram of computing device 100. Computing device 100 may include memory 202, processor 204, display 206, network interface 208, input device 210, and/or output module 212. Memory 202 includes any non-transitory computer-readable storage media for storing data and/or software that is executable by processor 204 and which controls the operation of computing device 100. In an embodiment, memory 202 may include one or more solid-state storage devices such as flash memory chips. Alternatively or in addition to the one or more solid-state storage devices, memory 202 may include one or more mass storage devices connected to the processor 204 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 204. That is, computer readable storage media includes non-transitory, volatile and non-volatile, removable and non-removable media implemented in any method or technology for storage of information such as computer-readable instructions, data structures, program modules, or other data. For example, computer-readable storage media includes RAM, ROM, EPROM, EEPROM, flash memory or other solid state memory technology, CD-ROM, DVD, Blu-Ray or other optical storage, magnetic cassettes, magnetic tape, magnetic disk storage or other magnetic storage devices, or any other medium which can be used to store the desired information and which can be accessed by computing device 100.

Memory 202 may store application 216 and/or functional respiratory imaging data 214 of one or more patients. Application 216 may, when executed by processor 204, cause display 206 to present user interfaces. Processor 204 may be a general-purpose processor, a specialized graphics processing unit (GPU) configured to perform specific graphics processing tasks while freeing up the general-purpose processor to perform other tasks, and/or any number or combination of such processors. Display 206 may be touch sensitive and/or voice activated, enabling display 206 to serve as both an input and output device. Alternatively, a keyboard (not shown), mouse (not shown), or other data input devices may be employed. Network interface 208 may be configured to connect to a network such as a local area network (LAN) consisting of a wired network and/or a wireless network, a wide area network (WAN), a wireless mobile network, a Bluetooth network, and/or the internet. For example, computing device 100 may receive functional respiratory imaging data, DICOM imaging data, computed tomographic (CT) image data, or other imaging data, of a patient from an imaging workstation 150 and/or a server, for example, a hospital server, internet server, or other similar servers, for use during surgical ablation planning. Patient functional respiratory imaging data may also be provided to computing device 100 via a removable memory 202. Computing device 100 may receive updates to its software, for example, application 216, via network interface 208. Computing device 100 may also display notifications on display 206 that a software update is available.

Input device 210 may be any device by means of which a user may interact with computing device 100, such as, for example, a mouse, keyboard, foot pedal, touch screen, and/or voice interface. Output module 212 may include any connectivity port or bus, such as, for example, parallel ports, serial ports, universal serial busses (USB), or any other similar connectivity port known to those skilled in the art.

Application 216 may be one or more software programs stored in memory 202 and executed by processor 204 of computing device 100. During a planning phase, application 216 guides a clinician through a series of steps to identify a target, size the target, size a treatment zone, and/or determine an access route to the target for later use during the procedure phase. In some embodiments, application 216 is loaded on computing devices in an operating room or other facility where surgical procedures are performed, and is used as a plan or map to guide a clinician performing a surgical procedure, but without any feedback from ablation device 130 used in the procedure to indicate where ablation device 130 is located in relation to the plan. In other embodiments, system 10 provides computing device 100 with data regarding the location of ablation device 130 within the body of the patient, such as by EM tracking, which application 216 may then use to indicate on the plan where ablation device 130 is located.

Application 216 may be installed directly on computing device 100, or may be installed on another computer, for example, a central server, and opened on computing device 100 via network interface 208. Application 216 may run natively on computing device 100, as a web-based application, or any other format known to those skilled in the art. In some embodiments, application 216 will be a single software program having all of the features and functionality described in the present disclosure. In other embodiments, application 216 may be two or more distinct software programs providing various parts of these features and functionality. For example, application 216 may include one software program for use during the planning phase, and a second software program for use during the procedure phase of the microwave ablation treatment. In such instances, the various software programs forming part of application 216 may be enabled to communicate with each other and/or import and export various settings and parameters relating to the microwave ablation treatment and/or the patient to share information. For example, a treatment plan and any of its components generated by one software program during the planning phase may be stored and exported to be used by a second software program during the procedure phase.

Application 216 communicates with a user interface 218 that generates a user interface for presenting visual interactive features to a clinician, for example, on display 206 and for receiving clinician input, for example, via a user input device. For example, user interface 218 may generate a graphical user interface (GUI) and output the GUI to display 206 for viewing by a clinician.

The ablation device 130 includes a distal tip 137 which may be blunt or may include a tapered trocar tip for percutaneous insertion through tissue. The ablation device 130 includes a radiating antenna 135 which includes a proximal radiating section 135a and a distal radiating section 135b, which are configured to emit microwave energy into the tissue surrounding the radiating antenna 135. The radiating antenna 135 is coupled to, or otherwise extends from, a cable 140 which couples the radiating antenna 135 to the microwave generator 40. A cooling tube 134 is positioned relative to the radiating antenna 135 for passage of a cooling fluid pumped from cooling system 50 to cool the radiating antenna 135 during application of microwave energy. An outer jacket 132 defines a lumen 132a and surrounds both of the cooling tube 134 and the radiating antenna 135. The cooling tube 134 may be positioned adjacent and alongside the radiating antenna 135 within the lumen 132a of the outer jacket 132 or the radiating antenna 135 may be positioned within the cooling tube 134. In percutaneous applications, the ablation device 130 includes an outer jacket 132 formed of a rigid material so as to maintain it's shape during percutaneous insertion of the ablation device 130 through tissue to a target. Alternatively, in lung navigation applications, ablation device 130 may include an outer jacket 132 which is formed of a flexible or semi-rigid material capable of being navigated through a catheter to a target.

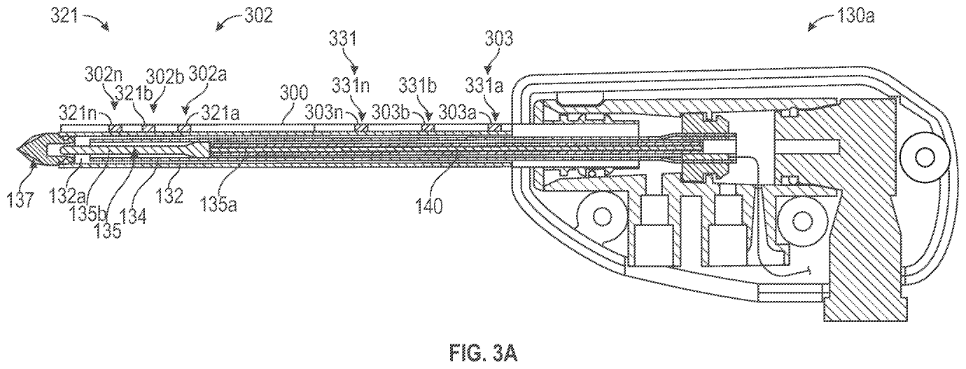

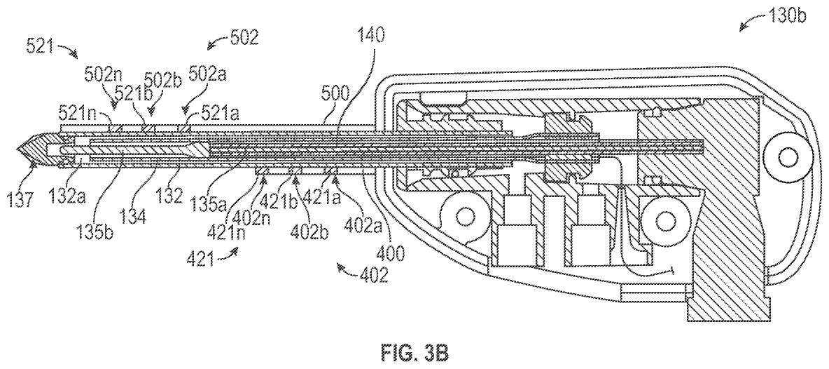

Referring now to FIGS. 3A and 3B, the ablation device 130a illustrated in FIG. 3A includes a single fiber 300 and the ablation device 130b illustrated in FIG. 3B includes a first fiber 500 and a second fiber 400. In aspects, one or more of fiber 300, fiber 400, and/or fiber 500 is embedded into the outer jacket 132 and/or may be coupled to the outer jacket 132. Alternatively, one or more of fiber 300, fiber 400, and/or fiber 500 may be positioned in the lumen 132a of the outer jacket 132 or on an outer surface of the outer jacket 132. In aspects, fiber 300, fiber 400, and/or fiber 500 may be retrofitted to an ablation device 130 by securing one or more of the fibers 300, 400, 500 to the outer jacket 132 via heat-shrink or other mediums. Respective proximal ends of the fibers 300, 400, 500 may be incorporated into a connector of the ablation device 130 which connects the ablation device 130 to a microwave generator (e.g., microwave generator 40), or alternatively, a separate independent connector may be utilized to connect the respective proximal ends of the fibers 300, 400, 500 to the microwave generator 40, computing device 100, or another component of system 10.

Referring specifically to FIG. 3A, fiber 300 includes a plurality of distal Bragg gratings 302a, 302b, . . . 302n (referred to collectively as distal Bragg gratings 302) etched into a distal portion of the fiber 300 in a region proximate the radiating antenna 135 of the ablation device 130a. Additionally, fiber 300 includes a plurality of proximal Bragg gratings 303a, 303b, . . . 303n (referred to collectively as proximal Bragg gratings 303) etched into a proximal portion of the fiber 300 in a region spaced apart from the radiating antenna 135 of the ablation device 130a.

The distal Bragg gratings 302 and the proximal Bragg gratings 303 reflect a narrow wavelength range called the Bragg wavelength. Each Bragg grating of the distal Bragg gratings 302 and the proximal Bragg gratings 303 includes periodic modulations in the core of the fiber 300 with spacing between each modulation. This changes the refractive index of the fiber 300 so that a single wavelength is reflected, while the rest of the light is transmitted down the fiber 300. The spacing between modulations changes when a distal Bragg grating 302 or a proximal Bragg grating 303 is subjected to a change in temperature and/or a force imparted thereon. This changes the refractive index of the Bragg grating 302, 303 and causes the Bragg wavelength to shift. Embodiments of the present disclosure use the shift in the Bragg wavelength to determine a temperature or motion of a component (e.g., cable 140).

The distal Bragg gratings 302 include a plurality of distal reflection points 321a, 321b, . . . 321n (referred to collectively as distal reflection points 321) and the proximal Bragg gratings 303 include a plurality of proximal reflection points 331a, 331b, . . . 33 In (referred to collectively as proximal reflection points 331) written into the fiber 300 at periodic spacing “Λ.” As the fiber 300 undergoes mechanical strain (e.g., a change in length) due to temperature and pressure changes, the spacing A is modified due to stretching or contraction of the fiber 300. The effects of changes in temperature on the fiber 300 is quantified by the computing device 100 by measuring the wavelength shift in light reflected by the distal reflection points 321 and the proximal reflection points 331 based on the following equation:

$$\frac{Δ λ}{{λ}_{0}} = k * ε + {α}_{δ} * Δ T \qquad ( 1 )$$

In equation (1), Δλ is the wavelength shift, λ0 is the base wavelength, k is a gage factor, which is a difference between 1 and a photo-elastic coefficient, ρ, ε is strain, ΔT is a telemetry change, and αδ is a change of the refraction index.

In this manner, fiber 300 is configured to transmit at least one wavelength of light and the Bragg gratings 302, 303 are configured to reflect at least one wavelength of light. Thus, the light transmissive properties, namely transmittance, of the fiber 300 along its length corresponds to a set of physical parameters of the ablation device 130 or the environment in which it is positioned (e.g., temperature of tissue).

The computing device 100 includes a fiber grating demodulator, which demodulates the reflected light transmitted through fiber 300 using a demodulation technique to obtain the changes in wavelength. Demodulation techniques include wavelength division multiplexing (WDM), optical time domain reflectometry (OTDM), optical frequency domain reflectometry (OFDM), and code correlation techniques that incorporate aspects of OTDM and OFDM. According to the OTDR technique, a narrow light pulse is generated by a light source and is transmitted through the optical fiber 300 to the distal Bragg gratings 302 and the proximal Bragg gratings 303. The reflected or backscattered light is analyzed to determine multiple telemetry values (e.g., temperatures, motion properties, etc.). The locations corresponding to each of the telemetry values (e.g., temperatures, motion properties, etc.) may be determined by monitoring the time it takes the reflected or backscattered light to return to the photodetector. Thus, the computing device 100 may distinguish the light reflected from the distal Bragg gratings 302 from the light reflected from the proximal Bragg gratings 303 by factoring the time it takes the light to return to the photodetector.

The telemetry measurements (e.g., temperature measurements) calculated by the computing device 100 using the light reflected from the distal Bragg gratings 302 may include noise, for example, noise generated by the pump 55 of the cooling system 50, and therefore may be an inaccurate representation of the actual temperature of the region. To address this issue, the computing device 100 calculates telemetry measurements corresponding to the motion induced by the pump 55 (e.g., motion imparted upon the cable 140) based on the light reflected from the proximal Bragg gratings 303, which as described above, are spaced apart from the distal Bragg gratings 302 outside of the ablation zone. Once both telemetry measurements are calculated, the computing device 100 is configured to filter the first telemetry measurements (e.g., temperature measurements) by subtracting the telemetry measurements calculated based on the proximal Bragg gratings 303 from the telemetry measurements calculated based on the distal Bragg gratings 302. The resulting filtered calculations correspond to a more accurate temperature value associated with the distal portion of the ablation device 130 and its surrounding tissue, which can be utilized by the computing device 100 to control the output of the generator 40 and/or to generate a simulation of an ablation zone for display to a clinician. In aspects, the above-described telemetry measurements calculated by the computing device 100 are compared to expected values within a predetermined operating range, and if the telemetry measurements fall outside of the predetermined operating range, the computing device 100 determines that a component is not operating normally and may issue a corresponding notification to the user.

Referring specifically to FIG. 3B, ablation device 130b is similar to ablation device 130a described above, but includes two distinct fibers (e.g., a first fiber 500 and a second fiber 400) instead of the single fiber 300 of ablation device 130a. The first fiber 500 includes a plurality of distal Bragg gratings 502a, 502b, . . . 502n (referred to collectively as distal Bragg gratings 502) etched into a distal portion of the first fiber 500 in a region proximate the radiating antenna 135 of the ablation device 130b. The distal Bragg gratings 502 include a plurality of distal reflection points 521a, 521b, . . . 521n (referred to collectively as distal reflection points 521) written into the first fiber 500 at periodic spacing “Λ1.” The second fiber 400 includes a plurality of proximal Bragg gratings 402a, 402b, . . . 402n (referred to collectively as proximal Bragg gratings 402) etched into a proximal portion of the second fiber 400 in a region spaced apart from the radiating antenna 135 of the ablation device 130b along the longitudinal axis of the ablation device 130b. The proximal Bragg gratings 402 include a plurality of proximal reflection points 431a, 431b, . . . 431n (referred to collectively as proximal reflection points 431) written into the second fiber 400 at periodic spacing “Λ2.” “Λ1” may be the same as or different from “Λ2.” In an aspect, a distal end of the second fiber 400 is longitudinally spaced apart from a distal end of the first fiber 500, along a longitudinal axis of the ablation device 135, such that the measurements acquired from the plurality of proximal Bragg gratings 402 are not impacted by the energy radiation exerted by the radiating antenna 135. As illustrated in the example of FIG. 3B, a distal end of the second fiber 400 is disposed proximal to a distal end of the first fiber 500, proximal to the plurality of distal Bragg gratings 502, and proximal to the distal radiating section 135b of the radiating antenna 135.

The distal Bragg gratings 502 and the proximal Bragg gratings 402 reflect a narrow wavelength range called the Bragg wavelength. Each Bragg grating of the distal Bragg gratings 502 and the proximal Bragg gratings 402 includes periodic modulations in the core of the first fiber 500 and the second fiber 500, respectively, with spacing between each modulation. The spacing between modulations changes when a distal Bragg grating 502 of the first fiber 500 is subjected to a change in temperature or physical characteristics such as motion imparted thereon. Likewise, the spacing between modulations changes when a proximal Bragg grating 402 of the second fiber 400 is subjected to a change in temperature or physical characteristics such as motion imparted thereon. This changes the refractive index of the Bragg grating 502, 402 and causes the Bragg wavelength to shift. Embodiments of the present disclosure use the shift in the Bragg wavelength to determine a temperature, motion, or other measurements or properties. The computing device 100 is configured to calculate a filtered telemetry measurement (e.g., a filtered temperature measurement) of the distal portion of the ablation device 130b in the same manner as described above with respect to the ablation device 130a, utilizing the telemetry measurements of the first fiber 500 and the telemetry measurements of the second fiber 400.

The temperature measurements sensed by computing device 100 may be displayed and/or may be used to control aspects of the system 10, for example, to control output of the microwave generator 40. In embodiments, the data is extrapolated during application of microwave ablation energy to simulate the volumetric geometry of the ablation zone as the ablation procedure progresses.

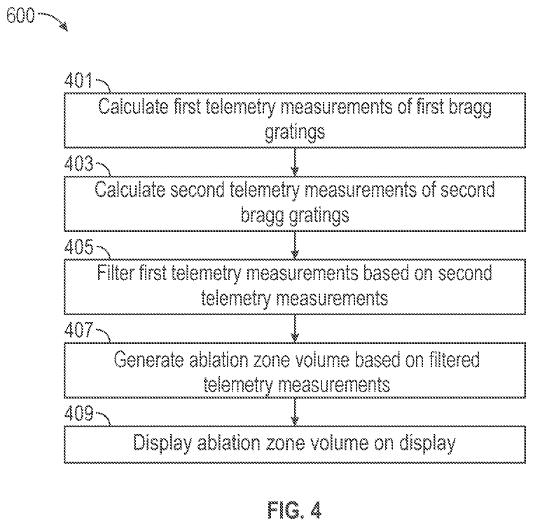

Turning to FIG. 4, a method for generating filtered telemetry measurements is illustrated and described as method 600. Method 600 is described as being executed by computing device 100, but some or all of the steps of method 600 may be implemented by one or more other components of the system 10, alone or in combination. Additionally, although method 600 is illustrated and described as including specific steps, and is described as being carried out in a particular order, it is understood that method 600 may include some or all of the steps described and may be carried out in any order not specifically described.

Method 600 begins at step 401 where computing device 100 calculates first telemetry measurements of first Bragg gratings. For aspects of an ablation device that includes a single fiber (e.g., ablation device 130a), step 401 includes calculating temperature measurements of distal Bragg gratings 302 of a fiber 300. For aspects of an ablation device that includes two fibers (e.g., ablation device 130b), step 401 includes calculating temperature measurements of distal Bragg gratings 502 of a first fiber 500.

In step 403, computing device 100 calculates second telemetry measurements of second Bragg gratings. For aspects of an ablation device that includes a single fiber (e.g., ablation device 130a), step 403 includes calculating motion induced upon a portion of the ablation device 130a by a pump 55 of a cooling system 50 as measured by light reflected from proximal Bragg gratings 303 of the fiber 300. For aspects of an ablation device that includes two fibers (e.g., ablation device 130b), step 403 includes calculating motion induced upon a portion of the ablation device 130b by a pump 55 of a cooling system 50 as measured by light reflected from proximal Bragg gratings 402 of a second fiber 400.

In step 405, computing device 100 filters the first telemetry measurements based on the second telemetry measurements. In aspects, step 405 may include subtracting the second telemetry measurements, which correspond to motion induced from a pump 55 (e.g., motion imparted upon cable 140), from the first telemetry measurements, which correspond to temperature measurements of a radiating antenna 135.

In aspects, method 600 further includes step 407, where the computing device 100 generates a simulation of an ablation volume based on the filtered temperature measurements, and step 409, where the simulated ablation zone may be displayed on a display device for viewing by a clinician.

Although embodiments have been described in detail with reference to the accompanying drawings for the purpose of illustration and description, it is to be understood that the inventive processes and apparatus are not to be construed as limited thereby. It will be apparent to those of ordinary skill in the art that various modifications to the foregoing embodiments may be made without departing from the scope of the disclosure.

What is claimed is:

1. An ablation system comprising:
an ablation device including:
a radiating antenna configured to apply microwave ablation energy to a target within a patient to ablate the target;
a cable extending from the radiating antenna and configured to connect the radiating antenna to a microwave generator;
a first fiber including first Bragg gratings formed along a length of the first fiber proximate the radiating antenna and configured to reflect light therefrom; and
a second fiber including second Bragg gratings formed along a length of the second fiber and longitudinally spaced apart from the first Bragg gratings along a longitudinal axis of the ablation device and configured to reflect light therefrom;
a cooling system including a pump configured to pump a cooling fluid to the ablation device to cool the ablation device; and
a computing device including a processor and memory storing instructions which, when executed by the processor, cause the computing device to:
calculate first telemetry measurements associated with the radiating antenna based on the reflected light from the first Bragg gratings;
calculate second telemetry measurements associated with the cooling system based on the reflected light from the second Bragg gratings; and
filter the first telemetry measurements based on the second telemetry measurements.

2. The ablation system of claim 1, wherein the computing device is configured to filter the first telemetry measurements by subtracting the second telemetry measurements from the first telemetry measurements.

3. The ablation system of claim 1, wherein the second telemetry measurements correspond to noise generated by the pump of the cooling system.

4. The ablation system of claim 1, wherein the first Bragg gratings are etched into the first fiber and the second Bragg gratings are etched into the second fiber.

5. The ablation system of claim 1, wherein the ablation device is a flexible microwave ablation catheter configured to be navigated through a patient's luminal network.

6. The ablation system of claim 1, wherein the ablation device is a rigid microwave ablation device configured to be percutaneously inserted through tissue.

7. The ablation system of claim 1, wherein the first telemetry measurements correspond to a temperature of the radiating antenna and the second telemetry measurements correspond to motion of the cable.

8. An ablation system comprising:
an ablation device including:
a radiating antenna configured to apply microwave ablation energy to a target within a patient to ablate the target;
a cable extending from the radiating antenna and configured to connect the radiating antenna to a microwave generator; and
a fiber including:
first Bragg gratings formed along a length of the fiber proximate the radiating antenna and configured to reflect light therefrom; and
second Bragg gratings formed along a length of the fiber and spaced apart from the first Bragg gratings and configured to reflect light therefrom;
a cooling system including a pump configured to pump a cooling fluid to the ablation device to cool the ablation device; and
a computing device including a processor and memory storing instructions which, when executed by the processor, cause the computing device to:
calculate first telemetry measurements associated with the radiating antenna based on the reflected light from the first Bragg gratings;
calculate second telemetry measurements associated with the cooling system based on the reflected light from the second Bragg gratings; and
filter the first telemetry measurements based on the second telemetry measurements.

9. The ablation system of claim 8, wherein the computing device is configured to filter the first telemetry measurements by subtracting the second telemetry measurements from the first telemetry measurements.

10. The ablation system of claim 8, wherein the second telemetry measurements correspond to noise generated by the pump of the cooling system.

11. The ablation system of claim 8, wherein the first Bragg gratings and the second Bragg gratings are etched into the fiber.

12. The ablation system of claim 8, wherein the ablation device is a flexible microwave ablation catheter configured to be navigated through a patient's luminal network.

13. The ablation system of claim 8, wherein the ablation device is a rigid microwave ablation device configured to be percutaneously inserted through tissue.

14. The ablation system of claim 8, wherein the first telemetry measurements correspond to a temperature of the radiating antenna and the second telemetry measurements correspond to motion of the cable.

15. A method for filtering telemetry measurements comprising:
receiving reflected light from first Bragg gratings and reflected light from second Bragg gratings;
calculating first telemetry measurements associated with a radiating antenna based on the reflected light from the first Bragg gratings;
calculating second telemetry measurements associated with a cooling system based on the reflected light from the second Bragg gratings; and
filtering the first telemetry measurements based on the second telemetry measurements.

16. The method of claim 15, wherein filtering the first telemetry measurements based on the second telemetry measurements includes subtracting the second telemetry measurements from the first telemetry measurements.

17. The method of claim 15, wherein receiving reflected light from first Bragg gratings and reflected light from second Bragg gratings includes receiving reflected light from first Bragg gratings formed in a first fiber and receiving reflected light from second Bragg gratings formed in a second fiber.

18. The method of claim 15, wherein calculating first telemetry measurements associated with a radiating antenna based on the reflected light from the first Bragg gratings includes calculating a temperature of the radiating antenna.

19. The method of claim 15, wherein calculating second telemetry measurements associated with a cooling system based on the reflected light from the second Bragg gratings includes calculating motion induced by a pump of the cooling system.

20. The method of claim 15, further comprising:
generating an ablation zone volume based on the filtered measurements; and
displaying the ablation zone volume on a display.
